(19) United States
(12) Patent Application Publication
(10) Pub. No.: US 2009/0069876 A1
(43) Pub. Date: Mar. 12, 2009

(54) System and Method for thermally Treating Tissues

(75) Inventors: Shaul Shohat, Kfar Haoranim (IL); Roni Shabat, Kibutz Yizrael (IL); Elazar Segal, Maccabim (IL); Adrian Paz, Petach Tikva (IL)

Correspondence Address: Avia Kafri, 22Shderot Chen St., Tel Aviv, 64166, IL

(73) Assignee: Biospiral Ltd., Nazareth (IL)

(21) Appl. No.: 12/087,469

(22) PCT Filed: Jan. 9, 2007
(86) PCT No.: PCT/IL07/00029

§ 371 Date: Jul. 8, 2008

Related U.S. Application Data
(60) Provisional application No. 60/757,015, filed on Jan. 9, 2006.

(51) Int. Cl. A61F 7/12

(52) U.S. Cl. 607/105; 606/27

(57) ABSTRACT

A system and method are provided for thermally treating tissues in percutaneous surgery or endoluminal treatments. The system consists of at least one catheter for thermally treating a tissue, hydraulic unit for circulating fluids through the catheters and a control station by which an operator is displayed with, and inputs and/or modifies, working parameters of the system. The catheters have an operational face, which is optionally expandable and encloses a lumen through which the fluids are circulated. The method disclosed implements cooling of the targeted tissue prior to its heating. For heating and or cooling the tissues, the operational face of a catheter is pressed against a surface of the targeted tissue while heat emitted from or conducted to the tissue is respectively transferred into or from the circulated fluids.

### FIELD OF THE INVENTION

The present invention relates in general to application of thermal energy to tissues in percutaneous surgery and endoluminal therapy. More particularly the present invention relates to devices for thermally treating tissues providing for contact heating and/or cooling the tissue by means of fluids.

### BACKGROUND OF THE INVENTION

Devices used for treating interstitial tissues thermally (e.g., thermotherapy, tissue coagulation and ablation) operate employing direct electrical heating, irradiating with ultrasonic radiation, or electromagnetic radiation in the frequency ranges of radio frequency or microwave, or by laser during percutaneous surgery are known in the art. The heat transferred to the targeted tissue in such processes must compensate for the accumulated rates of competing dissipation processes of heat transferred to surrounding layers or tissues. Excessive heating power may lead to undesirable boiling of fluids and/or charring of the targeted tissue. On the other hand, insufficient heat will not produce the desired results of coagulation, tissue forming and/or ablation. Typically such treatments are considerably painful for the patient, requiring him/her to remain motionless for a long period and therefore involve general anesthesia.

Therefore a method and system providing for thermal treatment during percutaneous surgery and/or endoluminal therapy, in which the treated tissue is homogeneously heated and/or cooled such that hot/cold spots are respectively avoided; that necessitate only local anesthesia; do not require an infrastructure such as normally present in operating rooms; can be conducted in doctors' clinics, and therefore are more convenient to the patient as well as to the surgeon are called for.

### BRIEF DESCRIPTION OF THE DRAWINGS

FIG. 1 is a block diagram of a system for thermally treating a tissue according to the present invention;

FIG. 2 is a sectional view of the distal segment of a catheter for thermally treating a tissue (CTT) of the invention;

FIG. 3 is a sectional view of a CTT according to a preferred embodiment of the present invention;

FIG. 4 is a sectional view of the distal segment of a CTT according to another preferred embodiment of the present invention enclosing a trocard;

FIG. 5 is a sectional view of the distal segment of the same CTT shown in FIG. 4 enclosing a guide wire;

FIG. 6 is a sectional view of the distal segment of a CTT in accordance with another embodiment of the present invention;

FIG. 7 is a schematic layout of a kit of accessories laid in front of a sectional view of the distal end of a CTT according to a preferred embodiment of the present invention;

FIG. 8 schematically describes a surgeon applying the system of the invention for ablating a prostate;

FIG. 9 is a graph of temperatures measured within a tissue during a heating phase, which follows a freezing phase.

### DETAILED DESCRIPTION OF THE PRESENT INVENTION

In accordance with the present invention a system and method for thermally treating tissues in percutaneous surgery and/or endoluminal therapy are provided. A system of the invention includes the following units and items: (a) one or more catheters for thermally treating a tissue; (b) a hydraulic subsystem for delivering cooled and or heated fluids to, and evacuating them from these catheters; (c) control station for operating the system and displaying data related to the processes to the operator; and (d) accompanying accessories, such as introduction needles, dilator sheathes, trocards, guide wires and guiding rods, some of which are grouped in various combinations into prepackaged kits.

The method according to the present invention implements contact heating and/or cooling of a tissue such that heat is transferred from, or to, a fluid flowing through the catheters. Blood circulation to and from cooled or thawing tissue is significantly lower as compared to the respective rates in the same tissue subjected to normal temperatures. Furthermore, the efficiency of the thermal treatment applied increases as the temperature of the targeted tissue is considerably lowered prior to its heating. Therefore cooling and heating a tissue are successively applied at the same location within a tissue such that a heating phase follows a cooling phase or successive heating phases are interleaved with cooling phases. Alternatively, cooling and heating are applied concomitantly at different locations. For example, cooling a peripheral region of a concomitantly heated location, or heating the periphery of a concomitantly cooled location. Cooling and/or freezing are accomplished according to the present invention by conducting heat from the tissue to a fluid cooled to a temperature considerably lower than the body temperature, which is within a range of 5° C. down to a temperature closely above the freezing point of the fluid employed. Optionally, cooling is induced by common freezing means while the treated tissue is further or concomitantly contact heated or cooled by means of catheters through which fluid having respective temperatures is delivered. Heating of the tissue is accomplished according the invention by conducting heat from heated fluids pressurized into the catheter of the invention. Alternatively, heating is accomplished by means of common heating probes while the treated tissue is contact heated or cooled by one or more catheters of the invention as is further described infra.

System for Thermally Treating a Tissue

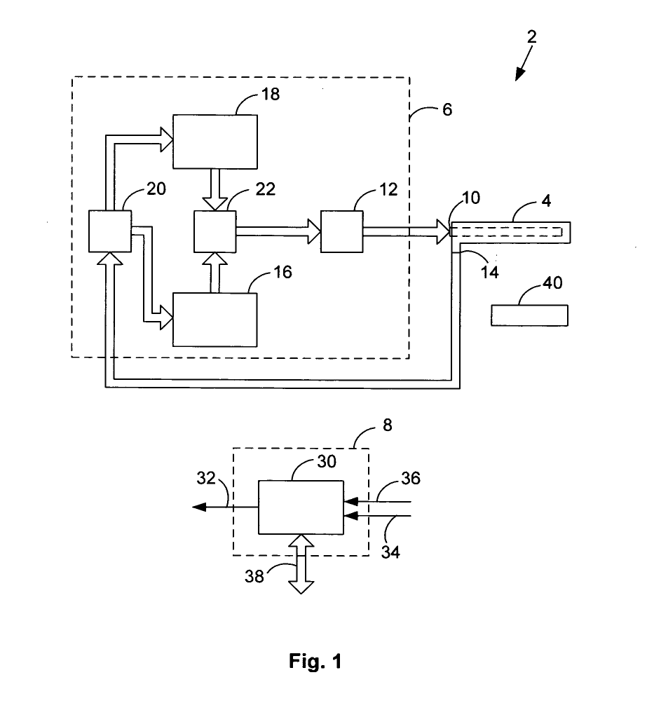

Reference is first made to FIG. 1 showing a block diagram of a system for thermally treating a tissue (STT) in accordance with the present invention. STT 2 consists of one or more catheters for thermally treating a tissue (CTT), such as CTT 4, hydraulic unit or subsystem 6 and local control station 8. Heated or cooled fluids are pressurized into inlet 10 disposed at the proximal end of CCT 4 by pump 12 and respectively evacuated through outlet 14 either to hot container 16 or to cold container 18. The fluids are circulated through the CTTs in a closed loop. The temperature of the fluids contained in both containers are controlled by means of temperature controlling devices having heating and/or cooling elements and temperature sensors respectively installed in both containers, not shown. Directional valve 20 provides for respectively selecting between the hot or cold containers. Directional valve 22 provides for respectively feeding pump 12 with either heated or cooled fluids. Additional directional valve or valves provide for directing the fluids pressurized by pump 12 to the other catheters, not shown. Optionally one or more containers are employed for storing fluids at different high and/or low temperatures.

Systems of the invention are characterized by their capability to sequentially circulate heated and/or cooled fluids through the same CTT and/or circulating a heated fluid through one CTT while a cooled fluid is concomitantly circulated through additional CTTs. For this purpose respective pumps, directional valves and temperature sensors and piping, not shown, are used between the inlet and the outlet of a pump and the respective containers. At this stage the fluids instead of being fed to the respective CTT are fed back to a respective container. When the temperature of such connected pump reaches the respective predefined value the directional valves are switched to a stage providing for feeding the respective CTT. Alternatively, fluids are stored in one container having an outlet channel that is respectively either heated or cooled to a predefined temperature. In such a case the fluids evacuated from a CTT are returned into this container and the respective directional valves may be avoided.

Control station 8 includes controller 30, a power supply unit and an operator interface unit, the latter two not shown. Controller 30 activates pump 12 and the directional valves as well as the heating and cooling devices, not shown, respectively installed in hot container 16 and cold container 18. Activating signals are transmitted from controller 30 to these devices by means of output discrete lines such as line 32 connecting between controller 30 and one of these devices. Analog and/or discrete signals received from various sensors embedded in hydraulic subsystem 8, not shown, such as temperature sensing devices, sensors for measuring the capacity of fluids fed into catheter 10 and/or evacuated from it, pressure sensors, as well as statuses of the pump and/or the directional valves and the level of fluids contained in each container are read into controller 30 by means of analog and discrete input lines such as line 34. Optionally signals generated by temperature sensors inserted within the targeted tissue, not shown, are input into controller 30 by means of analog input lines such as line 36. Link 38 to a remote computer, not shown, provides for uploading measurement and status data currently stored in the memory of controller 30 and/or for downloading working parameters and/or for remote programming of controller 30. The operator interface unit, not shown, provides for manually inputting or modifying working parameters of the system by, and for displaying process related data to, the operator and for activating the system and/or its processes.

Monitoring probe 40 provides for monitoring processes of the thermal treatment applied. Any common endoscopic means may be used for probe 40, which can be accommodated to the specific organ within the human body including the targeted tissue. Optionally, medical imaging systems such as ultrasonic, X rays or magnetic resonance imaging, are employed instead of, or in addition to, monitoring probe 40. Such endoscopic means are included in a non limiting list including means for performing tracheoscopy, bronchoscopy esophagoscopy, hysteroscopy, gastroscopy, urethroscopy, endoscopy of the vascular system, endoscopy of the small bowel, laparoscopy, thorascopy and arthroscopy.

Catheters and Accompanying Accessories

A CTT according to the invention is a slender body having an operational tip disposed at its distal end. The operational tip has a lumen and an operational face whose temperature closely equals the temperature of a fluid contained within this lumen. The external surface of a CTT except for its operational face is thermally insulated. Two fluid passages typically connect between the lumen of the operational tip and inlet and outlet apertures respectively disposed at the proximal end of the CTT. An optional tubular cavity is coaxially disposed within the CTT providing for threading of a guide wire through its lumen such that it distally and/or proximally extends from the respective open end of the cavity. Such cavity or alternatively one of the fluid passages optionally provides for introducing a guiding rod for pushing the CTT into a tissue. Catheters of the invention are characterized by their capability to withstand relatively high pressures and extreme high and low temperatures of the fluids circulated through them. The slender body can be made of elastic materials such that it is bendable providing for its introduction through curved or tortuous tracks as may be required for endoluminal therapy. A flexible CTT provided with a sharpened tip disposed at its distal end can be interstitially inserted by pushing a guiding rod inserted into its cavity. Following the placement of the CTT in the targeted location the guiding rod is removed and the CTT can be easily bended providing for relieving some of the pains involved.

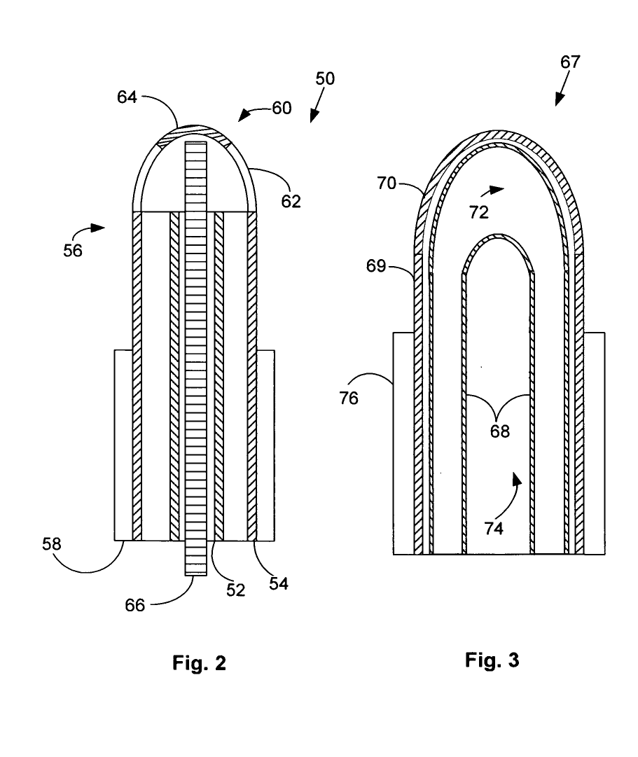

Reference is now made to FIGS. 2-6 in which sectional views of distal segments of CTTs in accordance with different embodiments of the present invention are respectively shown. CTT 50 has two concentric tubes, tube 52 and 54, providing for the delivering and evacuating of fluids into and from the lumen of operational tip 56 respectively. Operational tip 56 extends out of insulating sheath 58 enclosing tube 54. Cap 60 seals off the distal opening of tube 54, such that its lumen forms a continuum with the lumens of both tube 52 and 54. The face of cap 60 and the distal portion of the surface of tube 54 constitute the operational face of CTT 50. The tubes and cap 60 are normally made from plastic resins typically utilized for manufacturing elastic articles such as polyurethane or silicon fortified with additives for enhancing the thermal conductivity. Tubes and caps made of stainless steel are in accordance with the present invention. Optionally cap 60 or at least skirting segment 62 is made of a flexible material, such as polyurethane, and is therefore expandable such as a balloon. In such cases mainly the face of the expanded cap constitutes the operational face of the CTT, whereas the length of the segment of tube 54 distally extending from the insulating sheath is typically minimized. Segment 64 of cap 60 is optionally rigid such that guiding rod 66 when pressed against its inner face while being pushed into the lumen of tube 52 provides for introducing CTT 50 into a tissue. Optionally insulating sheath 58 is slidingly attached to tube 54 such that sliding insulating sheath 58 towards the proximal end of CTT 50 expands the operational face of CTT 50. CTTs of the invention including CTT 50 are provided at their proximal end, not shown, with an inlet and outlet connectors providing for connecting to piping of the hydraulic unit.

The insulating sheath enclosing CTTs of the invention provides for securing tissues and layers adjacent to the treated tissue from hazards of thermal injuries. The insulating sheath is made of any thermally insulating materials that are comprised of, or coated with, biocompatible materials such as Teflon. Similarly an optional gripping handle disposed close to the proximal end of the CTTs as well as the piping and their connectors are thermally insulated providing for conveniently being held or touched by an operator or a patient. Encapsulating the external surface of a CTT with a hollow vacuumed body or with a tubular body whose surface is thermally conducting, however its temperature is controlled, is in accordance with the present invention.

CTT 67, is schematic description of a preferred embodiment of the invention, is shown in FIG. 3. Collapsible tube 68, typically made of flexible material such as polyurethane or silicon rubber, is disposed folded within the lumen of external tube 69. Spherically shaped cap 70 seals off tube 69. Optionally cap 70 is conical featuring a sharpened tip at its distal end. Expanded segment 72 of tube 68 constitutes the lumen of CTT 67 through which the fluids are circulated and provides for enlarging the volume in which fluid is contained in close proximity to the operational face. Clearance 74 separating between the two segments of tube 68 constitutes the cavity of this CTT into which a guiding rod can be inserted. The segment of the surface of tube 69 distally extending from insulating sheath 76 and the surface of cap 70 constitute the operational face of this CTT. A CTT in accordance with another preferred embodiment of the present invention is similar to CTT 67 except that it does not have a cap at its distal end. The operational face of this CTT is the external surface of the segment of folded tube 68 when is slidingly moved to extend from the distal aperture of tube 69. Clearance 74 and collapsible tube 68 provide for introducing a trocard or guiding wire for the insertion of this CTT into a tissue.

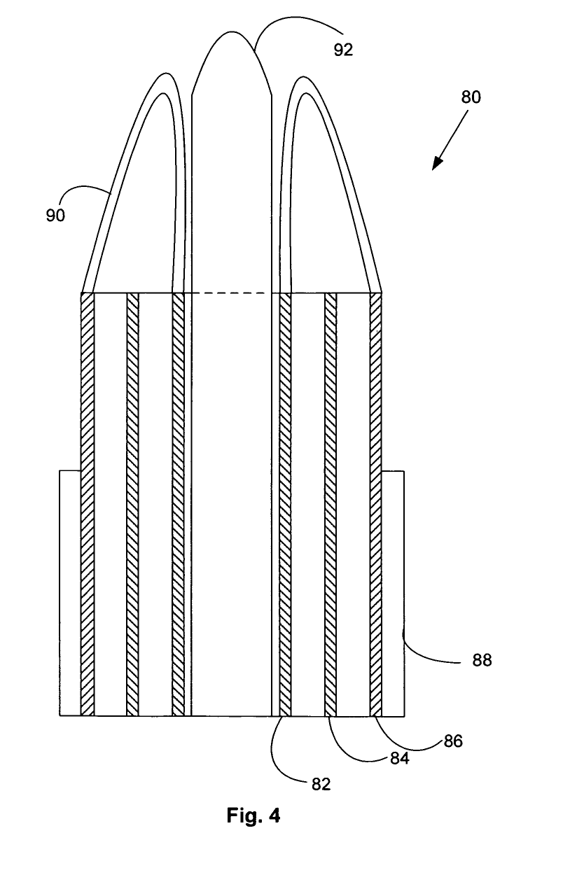

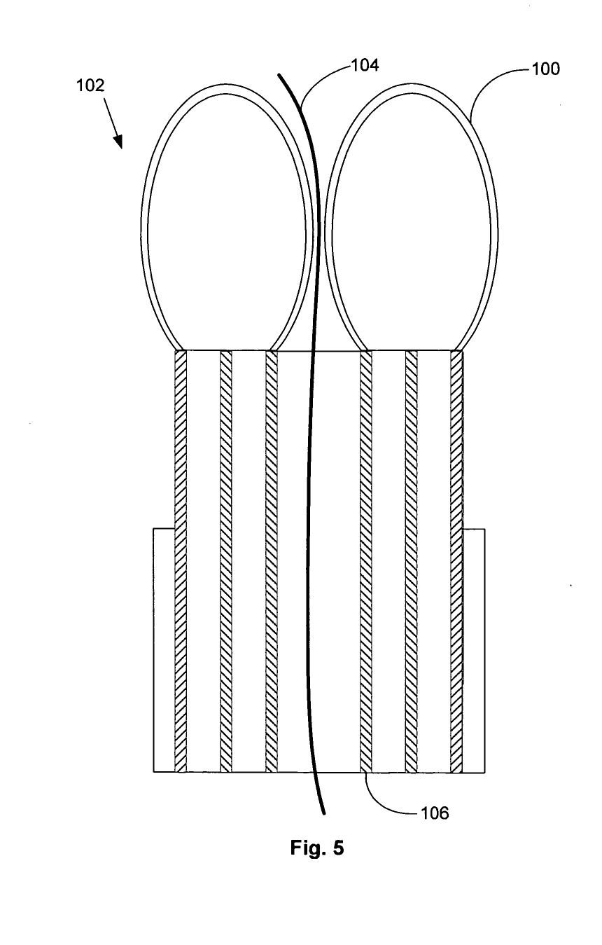

In FIGS. 4 and 5 a CTT in accordance with another preferred embodiment of the present invention is shown in two different stages respectively. CTT 80 has three concentric tubes, tube 82, 84 and 86 respectively. Insulating sheath 88 encloses tube 86 except for a short segment close to its distal end. Cap 90, is toroidal, seals off the distal apertures of tubes 84 and 86, such that its lumen forms a continuum with each lumen of both tubes. The lumen of tube 82 constitutes the cavity of CTT 80. Trocard 92 provides for closing the distal aperture of tube 82 thereby avoiding its blockage while CTT 80 is forced into tissue. Cap 100 of CTT 102 is expandable. Guide wire 104 passing along the lumen of tube 106 replaces the above-mentioned trocard and provides for directing the operational face of CTT 102 to the targeted location.

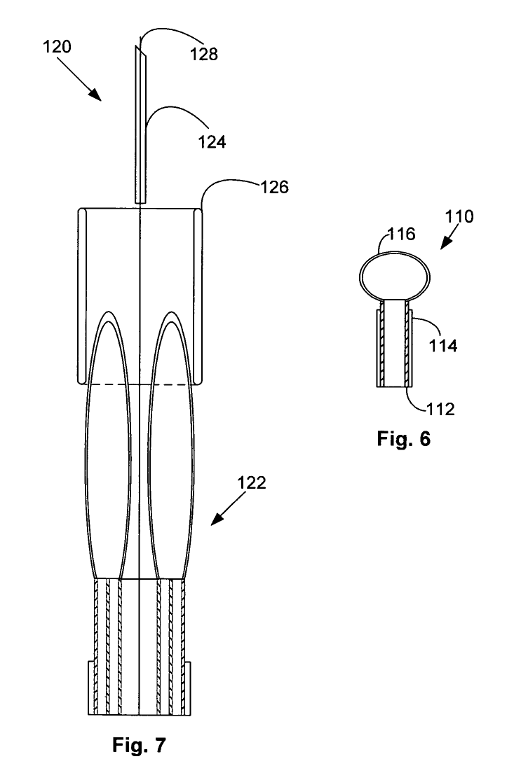

In FIG. 6 a CTT according to another embodiment of the present invention is shown. CCT 110 has tube 112 enclosed with insulating sheath 114. Expandable cap 116 is disposed at the distal end of CTT 110. Such CTT provides for transferring heat to or from a fixed quantity of fluid at a time. It is typically filled prior to each thermal phase with a fluid having a predefined temperature, which is evacuated after a period of time and/or prior to the following thermal phase for its refill. A STT including such a CTT is optionally equipped with a directional valve providing for directing the evacuated fluid to a container storing fluids having a compatible respective temperature.

Reference is now made to FIG. 7 schematically showing kit of accessories 120 according to a preferred embodiment of the present invention laid out in front of the distal end of CTT 122. Kit 120 includes needle 124 having a diameter of 0.2 up to 1 millimeter, dilation sheath 126 and guide wire 128 shown respectively passing through the lumens of the needle, the dilation sheath and CTT 122. In addition, kit 120 includes a trocard having an internal axial cavity for threading a guide wire through, not shown. Optionally, a kit of accessories includes introducing needles whose diameter is accommodated to encompass the distal end of a CTT. Optionally such introducing needles have a detachable proximal hub providing for their being removed off an inserted and placed CTT.

Operating CTTs

Introducing a CTT into a tissue according to the present invention is accomplished as follows: first a guide wire threaded through thin introducing needle is placed at the targeted location, then the tract originated by the introducing needle is widened by means of a dilating sheath while the guide wire is retained in place, then the CTT in which this guide wire is threaded through its cavity is introduced by forcing its tip into the tissues along the guide wire up to the targeted location. Alternatively, introduction needles accommodated to enclose the operational tip of the CTT including those having expandable caps, can be used for forcing a CTT into a tissue. CTTs such as those described with reference to FIGS. 2 and 3 to which reference is again made, which are optionally provided with a pointed cap, can be directly introduced into a tissue by forceing in by means of a guiding rod pressed into their cavity. Tissues disposed close to a surface of a lumen of an organ such as the uterine mocosa can be accessed for thermal treatment by introducing a CTT having an expandable cap to the uterus. A CTT having an expandable cap is applicable also for insertion into a tissue or a cavity in which the expanded cap provides for an enhanced contact heating due to the enlarged contacting surface. The pressure induced by the expanded cap onto surrounding surfaces fixes the operational tip of a CTT in place, and assists to reduce the blood circulation within the treated tissue. Therefore, such pressure provides for minimizing the heat dissipation to surrounding tissues thereby enhancing the efficiency of the thermal treatment applied.

Normally such introduction and placement of a CTT are monitored by means of common medical imaging methods such as ultrasonic, X rays or magnetic resonance imaging, and/or other common endoscopic methods accommodated to the targeted area within the human body. Therefore properly placing the operational tip can be accomplished.

Operating the STTs

The thermal treatment according to the method of the present invention subjects the targeted tissue to repetitive thermal cycles. Each cycle is preferably composed of a cooling or freezing phase and one or more heating phases successively following it. The same CTT or CTTs are successively employed for both cooling and heating phases. Namely, the treated tissue is subjected to one or more cycles including intermittently repeated cooling phases and heating phases. The operator places a CTT within the targeted tissue or changes the location in which a CTT is placed within the tissue preferably prior to a cooling or freezing phase. Optionally, a multiplicity of CTTs placed at different locations within a tissue is either simultaneously or independently operated for cooling and or heating.

For cooling and/or heating a tissue onto which an operational face of a CTT of the invention has been pressed a fluid having a predefined temperature is pressurized into this CTT. Feeding the CTT with such fluid continues until one, some, or the first, of the following events occurs: (a) a temperature measured at a predefined location or locations within the treated tissue reaches a predefined threshold; (b) the time elapsed equals a predefined time interval, and/or (c) a predefined event is detected by the operator who visually monitors the targeted tissue by means of a monitoring probe. Such a predefined event is an expansion of an “ice ball” visualized by mean of an ultrasonic imaging of a frozen tissue. The predefined time intervals are in the range of a few minutes.

The rates of heat transferred to or from the targeted tissue according to the method of the present invention are controlled by controlling: (a) the area of the operational faces of the CTTs involved, such as by expanding catheters' caps or by proximally sliding an insulating sheath of a CTT thereby exposing an extended segment of the surface enclosing the fluid contained within the CTT, (b) selecting a temperature of the fluids fed into, or circulated through, the CTTs, (c) controlling the rate of flow of the fluids through the catheters, such as by varying the pressure of the fluid, and (d) improving the contact between the operational

Optionally a CTT of the invention is used in combination with a common freezing apparatus for freezing the tissue. Exemplary freezing apparatuses normally employed are of the type utilizing liquid nitrogen or the type in which cooling is effected by an expansion of gas such as argon. In such cases the CTTs employed have an expandable cap such as of the CTT described with reference to FIGS. 4 and 5 to which reference is again made. First a fluid at the respective predefined temperature is pressurized into the CTT whose cap is expanded to a predefined volume or at a predefined pressure and pressed against the surface of the tissue enclosing it. Then the freezing probe of a freezing apparatus is further introduced independently or through the cavity of another CTT to freeze the targeted tissue at a different location as known in the art. Heat is conducted to circumferential tissue from the fluid continuously fed into the CTT at the same pressure, or in the opposite direction, such that the tissue is frozen while its periphery is either heated or cooled respectively.

In order to heat tissue the temperatures of the heated fluid and the lengths of the heating time intervals are selected in accordance with the specific process to be applied (e.g. thermal forming, coagulation and/or ablation of the targeted tissue). Optionally, heating tissue is accomplished by means of a CTT of the invention in combination with a heating probe of a common heating apparatus such as a laser fiber. CTTs employed in such cases are similar to the CTT described with reference to FIGS. 4 and 5. The cap of the CTT is expanded to a predefined volume or at a predefined pressure; a fluid having a predefined temperature is then further pressurized into the CTT. The fluid is continuously fed at the same pressure while the heating probe is inserted independently or through the lumen of the cavity of another CTT to heat the targeted tissue as known. The periphery of the tissue heated by means of the heating probe is either contact heated or cooled in accordance with the temperature of the circulated fluid.

The temperature of tissue is monitored as is known, such as by means of thermocouples inserted into the targeted tissue at a vicinity, preferably close, to the operational face of a CTT. The heating and the cooling are further monitored by means of the monitoring probe and/or the imaging systems employed.

The controller of a STT automatically monitors the quantities of fluids entering and emerging each CTT. In cases in which these quantities differ and the difference exceeds a predefined threshold an alarm is automatically activated simultaneously with turning off the pump of the hydraulic subsystem thereby pressurizing fluid into a CTT is automatically stopped. Therefore hazards that might be caused by spilled fluids within a tissue, such as tissue dehydration or a spillage of a toxic fluid, during a thermal treatment according to the present invention are substantially minimized.

Any liquid that is biocompatible and the freezing point of which is considerably lower than 0° C. and the boiling temperature of which exceeds 45° C. can be utilized as an operative fluid according to the present invention. Suitable are aqueous solutions, due to their relatively high heat capacity. Preferable are aqueous solutions of non-toxic alcohols such as ethanol, which retain a liquid phase considerably below 0° C. down to −100° C. and lower. Similarly,

freezing point depressants such as some glycoprotein are preferable as well. Applicable are aqueous solutions of compositions normally utilized as anti freezing agents, such as dimethylsulfoxide (DMSO) or polyethylene glycol or concentrated aqueous salt solutions. Organic compositions or hydrocarbons having short carbon chains of 1-8 atoms, such mixtures and solutions whose freezing temperature is below 0° C., are less favorable due to toxicity. Pure aqueous solutions, such as saline are preferable for heating tissue. In cases in which the same fluid is employed both for heating and cooling its boiling temperature must exceed 450 and preferably 50° C.

Potential Applications

The system and method of the present invention provide for a variety of thermal treatment in subcutaneous surgery including but not limited to the following: ablating liver tumors in which CTTs are introduced through the abdominal wall, ablating prostate tumor, ablating the uterine mucosa (endometrium) in cases of excessive uterine bleeding, ablating benign prostate hyperplastic tissue close to the urethra mucosa, ablating esophageal tumors close to the esophageal mucosa, ablating dysplastic esophageal mucosa in cases of gastro-esophageal reflux, ablating endocardial tissue in cases of cardiac rhythm disorder such as atrial fibrillation, ablating endovascular atheromatous lesions, ablating the uterine cervix mucosa in cases of carcinoma in situ, remodeling the tissue close to the urethra or close to the neck of the urinary bladder in cases of urinary incontinence, remodeling tissues close to the lower esophageal sphincter in cases of gastro-esophageal reflux, etc.

### EXAMPLE 1

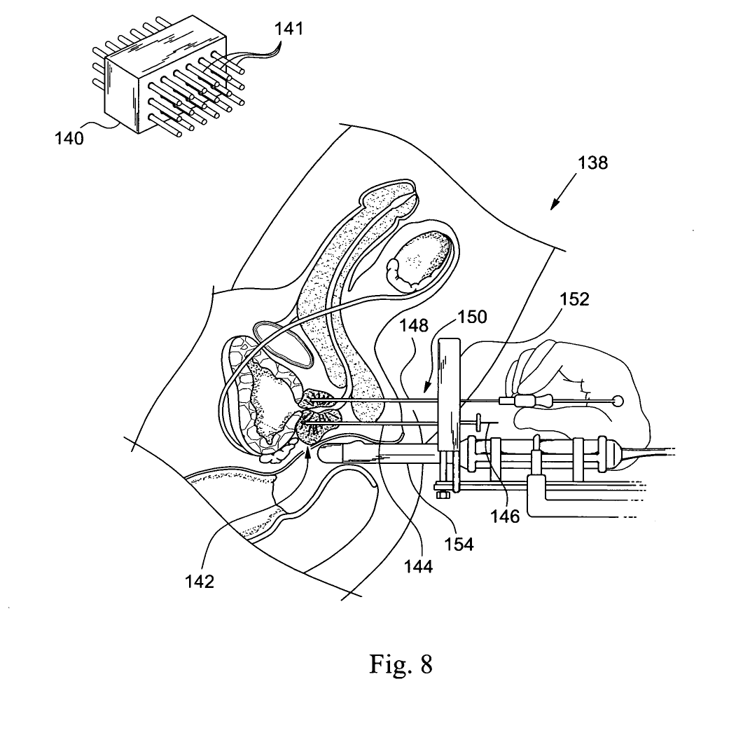

An exemplary process of thermal ablation of the prostate for treating prostate cancer is described herein below with reference to FIG. 8 in which patient 138 positioned in a lithotomy position is schematically shown. The patient legs are supported with stirrups as known. A number of CTTs 140 are housed in stand 141 ready to be inserted into prostate 142. Introducing needle 144 through which guide wire 146 is threaded and thermocouple 148 are introduced to the prostate through perineum 150. Two-dimensional grid 152 provides for aligning and properly placing the CTTs and/or thermocouples. Introduction of the CTTs and/or thermocouples is carried out while being monitored by means of trans rectal ultrasonic (TRUS) probe 154.

CTTs following their introduction and placement within the prostate are further connected to the piping of a STT, not shown, and phases of freezing followed heating the tissue are applied as described hereinabove. A freezing phase starts according to the method of the invention by first pressurizing cooled fluid to inflate the caps of the inserted CTTs and retaining them at the same pressure and at a temperature, which is lower than 0° C., for a period (up to a few minutes). The freezing process is monitored by means of the TRUS. Thereafter Alternatively, introducing needles capable for encompassing a CTT are employed and the use of the guide wires is avoided.

### EXAMPLE 2

An exemplary process of ablating the benign prostate hyperplasia (BPH) according to another preferred embodiment of the present invention is described below. The patient is positioned in a lithotomy position with legs supported with stirrups as described hereinabove in example 1. A CTT such as described with reference to FIG. 4 or 5 to which reference is again made is introduced through the working channel of a cystoscope inserted to the urethra. The introduction of a CTT is effected either by the aid of a guide wire or directly by employing an introducing needle. Such introduction is preferably monitored by means of a TRUS. Preferably three such CTTs are employed of which two are respectively placed within each lateral lobe of BPH and the third CTT is placed within the is middle lobe. Both phases of freezing and heating are performed whilst being monitored by means of a TRUS similarly to the same process described in example 1 hereinabove.

Alternatively CTTs having expandable cap and a cavity for the insertion of guide wires or trocards such as shown in FIG. 4 are employed. The freezing phase is carried out as described hereinabove. However, in addition to pressurizing a fluid into the cap of the CTT at the end of the freezing phase a heating probe, such as laser fiber or a RF heating probe, is inserted through the lumen of another CTT providing for ablating the tissue at another location as is known. Therefore the periphery of the ablated tissue is further heated or cooled in accordance with the temperature of the pressurized fluid. The cooling and heating phases are similarly monitored by means of a TRUS.

### EXAMPLE 3

Exemplary procedure for ablating the lining of the uterine cavity is hereinafter described with reference to FIG. 2 to which reference is again made. Cap 60 of CTT 50 is expandable to a relatively large volume its geometrical shape and sizes are preferably accommodated to match the uterine cavity. In accordance with the method of the present invention CTT 50 is introduced into the uterine cavity through the vagina. Initially, cooled fluid is pressurized into the CTT at a predefined pressure while ultrasound imaging monitors the process. Freezing is also monitored by means of a few thermocouples that have been inserted into the uterine wall at predefined locations. After a few minutes when the temperature measured by the thermocouples approach a predefined threshold the freezing process is stopped. Fluid heated to 80° C. is pressurized into the CTT at the same pressure and the heating phase is started. The heating phase continues while the process is visually monitored by means of the ultrasound imaging and while the temperatures measured by the embedded thermocouples are inspected for a few minutes. The heating phase is stopped after a few minutes.

### EXAMPLE 4

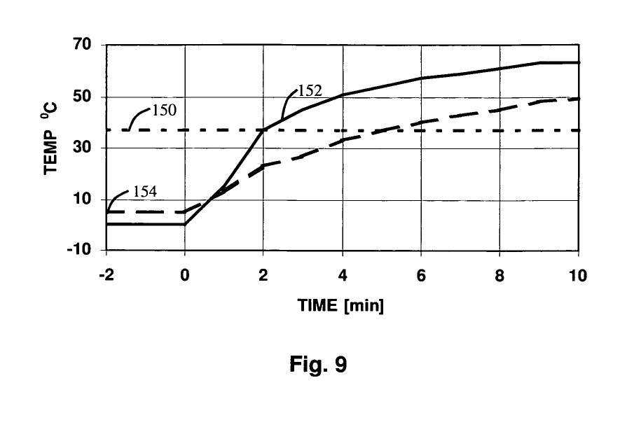

Laboratory experiments have been conducted for the purpose of demonstrating the method for controlling the rate in which heat is transferred to and/or from a tissue by contact heating or cooling according to the present invention. An exemplary experiment is hereby described with reference to FIGS. 4 and 9. A chunk of beef of a few centimeters (cm) cut from the biceps femoris was removed immediately following its severing and further stored in a thermal bath containing isotonic saline solution at 37° C. Experiments began less than 3 hours later. This chunk placed within the thermal bath simulated the targeted tissue for a thermal treatment according to the present invention. Two CTTs as described hereinabove with reference to FIG. 4 were employed for first freezing the tissue and heating it thereafter. The CTTs have a sharpened tip disposed at their distal end. The external diameter of both CTTs is 1.8 mm and they consist of three coaxial tubes made of stainless steel. An insulating sheath made of Teflon is slidingly attached to the surface of the external tube. A trocard having a diameter of 0.5 mm provided for inserting the CTTs into the targeted locations within the tissue, which were spaced apart by about 1 cm and respectively disposed about 3 cm below the topside of the chunk of beef. The fluid employed both for heating and cooling was a water-based solution of ethanol at a concentration of 50%. One junction of the first thermocouple was placed between the two CTTs within the tissue at about the same depth, at a distance of about 4-5 mm from each CTT. A second thermocouple measured the temperature at a distance of 1 cm from both CTTs and at the same depth of 3 cm. The temperatures read by means of these thermocouples as well as the temperatures of the thermal bath and the fluid circulated through the CTTs were continuously measured and recorded. The tissue was further monitored by means of a duplex—ultrasound imaging device (USID) the imaging probe of which was pressed against the side face of the chunk immersed in the saline solution.

Initially, two CTTs were introduced into the tissue and the insulating sheaths enclosing tube 84 of each CTT were respectively pulled out of the tissue each by 2.5 cm; thereby the operational faces of the CTTs were expanded. Then fluid at −10° C. was pressurized at a pressure of 13 atmospheres (atm) into the inlet apertures of both CTTs. The time elapsed from the moment in which the pump was turned on until a freezing phase has been completed was approximately 10 minutes (min). The temperature decreased slowly in the first minute and then dropped in considerable slopes as read by both thermocouples along the following 4 min. Then the first thermocouple reached 0° C. after additional 8 min whereas the second reached 5° C. about 2 min later. Freezing occurred after about 12 min. The criteria for the end of the freezing phase were the following: (a) an almost constant reading of the first thermocouple for 20 seconds of a temperature of 0° C.; (b) the expansion of ice balls to have a diameter of about 5 mm extending outwards from the distal ends of both CTTs as was visualized by means of the USID.

By the conclusion of this cooling phase a heating phase in which the two CTTs were simultaneously employed, was applied. Both CTTs were fed by the same fluid, which has been heated to 80° C., and pressurized at 13 atm. Plot 150 shown in FIG. 9 is the temperature profile of the thermal bath. Plots 152 and 154 are the temperature profiles as were measured by means of the first and second thermocouples respectively. The tissue at the targeted locations has reached the temperature of 45° C. after 3 min and the temperature of 50° C. after about 3.7 min. The second thermocouple starts at a higher temperature and lags behind the first one along the heating phase as it is disposed at a greater distance from both CTTs.

1. A system for thermally treating a tissue (STT), said system comprising
at least one catheter for thermally treating a tissue (CTT), wherein said CTT comprises
a slender tubular body having a proximal end and a distal end;
an operational face disposed at said distal end;
a lumen enclosed within said operational face;
an inlet aperture and an outlet aperture disposed at said proximal end for feeding a fluid into said lumen, and wherein two fluid passages connect between one said lumen and said inlet aperture and said outlet aperture respectively each, and
a hydraulic unit for circulating said fluid through said lumen, wherein said hydraulic unit comprises
a pump connectable with said CTT for feeding said fluid into said lumen;
at least one temperature controlling device for controlling the temperature of said fluid at a predefined temperature, and wherein said predefined temperature is changeable within a range of temperatures starting at −100° C. up to 100° C., and
a controller electrically connected to said pump and to said at least one temperature controlling device respectively, for at least activating said pump to successively and intermittently feed said lumen with said fluid at one of said predefined temperatures for a while and further feed said lumen with said fluid at another of said predefined temperatures for a while thereafter.

2. A STT as in claim 1, wherein said CTT further comprises a tubular cavity coaxially disposed within said slender tubular body.

3. A STT as in claim 1, wherein said pump is connectable with said inlet aperture.

4. A STT as in claim 1, wherein said hydraulic unit further comprises a pressure sensor for measuring a pressure of said fluid.

5. A STT as in claim 1, wherein said hydraulic unit further comprises
means for measuring quantities of said fluid fed into said lumen, and
means for measuring quantities of fluid evacuated from said lumen.

6. A STT as in claim 1, wherein said operational face is expandable.

7. A STT as in claim 1, further comprising a sensor for measuring a temperature of said tissue electrically connected to said controller.

8. A STT as in claim 1, further comprising a monitoring probe for monitoring at least a portion of said tissue.

9. A STT as in claim 6, wherein a segment of said operational face conforms a portion of a lumen of an organ when said operational face is being expanded.

10. A STT as in claim 2, in combination with a heating probe of a heating apparatus, wherein said tubular cavity accommodated for the insertion of said heating probe trough its lumen.

11. A STT as in claim 2, in combination with a freezing probe of a freezing apparatus, wherein said tubular cavity accommodated for the insertion of said heating probe trough its lumen.

12. A method for thermally treating a tissue by first cooling said tissue and further heating said tissue thereafter, wherein said cooling and said heating each comprise the steps of
a. placing a segment of the operational face of a catheter for thermally treating a tissue (CTT) pressed against a portion of said tissue;
b. circulating a fluid having a predefined temperature through said CTT, and
wherein said CTT comprises a slender tubular body having a proximal end and a distal end, and wherein said CTT has an operational face enclosing a lumen through which sad circulated fluid passes, and wherein said operational face disposed at said distal end.

13. A method as in claim 12, wherein said cooling comprises freezing.

14. A method as in claim 12, wherein said CTT further comprises a tubular cavity coaxially disposed within said slender body, wherein said cavity has two apertures one of which disposed at said distal end and the other at said proximal end.

15. A method as in claim 13, wherein said freezing applied by means of a freezing probe of a freezing apparatus inserted through the cavity claimed in claim 14.

16. A method as in claim 14, wherein said heating applied by means of a heating probe of a heating apparatus inserted through said cavity.

17. A method as in claim 12, wherein said CTT is any of a plurality of CTTs placed in said tissue.

18. A method as in claim 12, further comprising a step of pressurizing said fluid at a predefined pressure concomitantly with said circulating.

19. A method as in claim 12, further comprising expanding said operational face.

20. A method as in claim 12, further comprising comparing a quantity of fluid fed into said lumen with a quantity of fluid evacuated from said lumen.

21. A method as in claim 12, wherein said pressing is effected by pressurizing a fluid into said lumen.

22. A method as in claim 17 wherein said heating and said cooling are concomitantly effected by which said tissue is cooled by one of said CTTs the operational face of which disposed at one location within said tissue and another of said CTTs the operational face of which disposed at another location within said tissue.

23. A method as in claim 21, further comprising controlling the rates of said heating and said cooling by any activity selected from a group of activities consisting of expanding said operational face, contracting said operational face, distally extending said operational face, varying said predefined temperature, varying a pressure of said circulated fluid and any combination thereof.

24. A STT as in claim 1, wherein said fluid is any fluid selected from a group of fluids consisting of pure water solutions, aqueous solutions of salts, aqueous solutions of alcohols, aqueous solutions of anti-freezing agents, aqueous solutions of freezing point depressants, organic compositions whose respective freezing point is considerably below 0° C., and their respective boiling point exceeds 45° C., hydrocarbons having short carbon chains of 1-8 atoms, mixtures of said hydrocarbons, solutions of said hydrocarbons.

25. A catheter for thermally treating a tissue (CTT) comprising
a slender tubular body having a proximal end and a distal end;
an operational face disposed at said distal end;
a lumen enclosed within said operational face;
two apertures disposed at said proximal end, wherein two fluid passages connect between said lumen and said two apertures respectively each, and
an insulating sheath encloses a segment of the surface of said slender tubular body.

26. A CTT as in claim 25, wherein said thermal insulating sheath is slidingly attached to said surface.

27. A CTT as in claim 25, further comprising a tubular cavity coaxially embedded in said slender tubular body, wherein said cavity has an aperture disposed at said proximal end.

28. A CTT as in claim 27, wherein said tubular cavity has an aperture disposed at said distal end.

29. A CTT as in claim 25, wherein a segment of said operational face is expandable.

30. A CTT as in claim 25, wherein a segment of said fluid passage enclosed within a collapsible wall.

31. A CTT as in claim 25 further comprising a sharpened tip disposed at said distal end.

32. A CTT as in claim 25, wherein said slender tubular body is bendable.
